(19) United States
(12) Patent Application Publication
(10) Pub. No.: US 2020/0155265 A1
(43) Pub. Date: May 21, 2020

(54) Surgical Accessory and Methods of Using the Same

(71) Applicant: C-Clear, LLC, Midvale, UT (US)

(72) Inventors: Fredrick Brody, Bethesda, MD (US); Steve Eubanks, Winter Park, FL (US); Matt Penny, Holly Springs, NC (US)

(21) Appl. No.: 16/598,792

(22) Filed: Oct. 10, 2019

Related U.S. Application Data
(63) Continuation-in-part of application No. 16/573,616, filed on Sep. 17, 2019.
Application No. 16/573,616 is a division of application No. 15/418,022, filed on Jan. 27, 2017, now abandoned.
(60) Provisional application No. 62/288,143, filed on Jan. 28, 2016.

(51) Int. Cl. A61B 90/70; A61B 1/12; B08B 9/045

(52) U.S. Cl. CPC A61B 90/70; A61B 2090/701; B08B 9/045; A61B 1/122

(57) ABSTRACT

A minimally invasive device for cleaning surgical instruments includes a rod having first and second ends with a first sponge assembly fitted about the first end of the rod and a second sponge assembly fitted about the second end of the rod. At least one of the first and second sponge assemblies is removably secured to one of the respective first and second ends of the rod. Each of the first and second sponge assemblies is rotationally retained about the first or second ends of the rod such that the sponge assemblies are restrained from rotating relative to the rod.

### PRIORITY CLAIM

This application is a continuation-in-part of U.S. patent application Ser. No. 16/573,616, filed Sep. 17, 2019, which is a divisional of U.S. patent application Ser. No. 15/418,022, filed Jan. 27, 2017, which claims priority to U.S. Provisional Application Ser. No. 62/288,143, filed on Jan. 28, 2016, each of which is hereby incorporated herein by reference in its entirety.

### BACKGROUND OF THE INVENTION

### Field of the Invention

The present disclosure relates generally to devices used to clean both the trocar and the lens of a scope used in minimally invasive surgery. The present disclosure also relates to methods of cleaning both the trocar and the lens of a scope used in minimally invasive surgery by inserting a rod through the trocar and the foam end conforms to the inner cannula of the trocar.

### Related Art

The number of minimally invasive, robotic, and endoscopic procedures continue to increase since these types of procedures allow patients to heal faster with a shorter recovery time and a decreased risk for wound infections and hernias. These procedures entail small incisions to introduce instruments and scopes into various body cavities to perform a wide range of medical procedures that include ligating, cutting, suturing, and repairing tissue. However, these techniques require a continuous and clear image during the entire procedure. Typically, cannulas, trocars, or ports are placed through the small incisions and serve as portals to various body cavities. Due to the breadth of procedures, trocars are made of varying materials that range in length and diameter from one manufacturer to another.

Once the scope is inserted through a trocar, the scope lens may become obstructed due to smudging, condensation, or direct contact with pieces of tissue, bodily fluids, and condensation. These fluids include blood, bile, and fecal material. Also, pieces of tissue and fluid may be trapped within the trocars and collect on the lens as the scope is inserted into the body. Unfortunately, the field of view may be compromised numerous times during a procedure and prolong or hinder the progress of the procedure.

In order to provide the surgeon with a clear view, the scope needs to be removed from the body cavity, cleaned, and reinserted. During some procedures, it is not uncommon to remove and clean the scope 10-20 times which substantially increases the duration of the procedure and potentially leads to other complications. Thus, there is a need for a device that cleans the lens of the scope while the scope remains in a body cavity. As well, the device should concurrently clean the entire length of the trocar to ensure that tissue and fluid do not collect on the scope lens as it is inserted into the body.

The disclosed device and methods are directed to one or more of the problems set forth above and/or other problems of the prior art.

### SUMMARY OF THE INVENTION

In accordance with one aspect of the technology, a minimally invasive device for cleaning surgical instruments is provided, including a rod having first and second ends. A first sponge assembly can be fitted about the first end of the rod and a second sponge assembly fitted about the second end of the rod. At least one of the first and second sponge assemblies can be removably secured to one of the respective first and second ends of the rod. Each of the first and second sponge assemblies can be rotationally retained about the first or second ends of the rod such that the sponge assemblies are restrained from rotating relative to the rod.

In accordance with another aspect of the technology, a minimally invasive device for cleaning surgical instruments is provided, including a rod having first and second ends. A first sponge assembly can be fitted about the first end of the rod and a second sponge assembly can be fitted about the second end of the rod. At least one of the first and second sponge assemblies can be removably secured to one of the respective first and second ends of the rod. At least one end of the rod can include an indentation sized to receive one of the sponge assemblies therein. The indentation can have an outer surface recessed relative to an outer surface of an adjacent portion of the rod, the sponge assembly thereby being restrained from lateral movement relative to the rod. Each of the first and second sponge assemblies can be rotationally engaged about the first or second ends of the rod such that the sponge assemblies are restrained from rotating relative to the rod.

In accordance with another aspect of the technology, a minimally invasive device for cleaning surgical instruments is provided, including a rod having first and second ends. A first sponge assembly can be fitted about the first end of the rod and a second sponge assembly can be fitted about the second end of the rod. At least one of the first and second sponge assemblies can be removably secured to one of the respective first and second ends of the rod. Each of the first and second sponge assemblies can be rotationally retained about the first or second ends of the rod such that the sponge assemblies are restrained from rotating relative to the rod. At least one of the sponge assemblies can present differing first and second cleaning interfaces, each configured to be contacted by the surgical instrument.

### BRIEF DESCRIPTION OF THE DRAWINGS

The following drawings illustrate exemplary embodiments for carrying out the invention. Like reference numerals refer to like parts in different views or embodiments of the present invention in the drawings.

FIG. 1 is a perspective view of device according to the present disclosure;

FIG. 2 is another view of the device shown in FIG. 1;

FIG. 3 is a partially-sectioned, diagrammatic side view of an embodiment of a device constructed in accordance with principles of the present disclosure;

FIG. 4 is another view of the device shown in FIG. 3;

FIGS. 5A and 5B are partially-sectioned, diagrammatic side views of one end of a device constructed in accordance with principles of the present disclosure having a sponge that is screwed thereon;

FIG. 6 is a schematic of the sponge that can be used in an embodiment of a device constructed according to the present disclosure;

FIG. 7 is a schematic showing a threaded end of a rod according to the present disclosure;

FIG. 8 is a schematic of a device according to the present disclosure;

FIG. 9 is a schematic of a device according to the present disclosure;

FIG. 10 is a schematic of a device according to the present disclosure;

FIG. 11 is a perspective view of a baton in accordance with the present technology being used to clean an end of a surgical instrument;

FIG. 12A is a perspective view of a sponge assembly in accordance with another embodiment of the technology;

FIG. 12B is an exploded view of the sponge assembly of FIG. 12A;

FIG. 13A is a side view of an exemplary baton rod in accordance with an embodiment of the technology;

FIG. 13B is a sectional view of the rod of FIG. 13A, taken along section B-B of FIG. 13A;

FIG. 14A is a sectional view of an exemplary baton rod in accordance with an aspect of the technology;

FIG. 14B is a sectional view of another exemplary baton rod in accordance with an aspect of the technology;

FIG. 14C is a sectional view of another exemplary baton rod in accordance with an aspect of the technology;

FIG. 15A is a side view of an exemplary baton rod in accordance with an embodiment of the technology; and

FIG. 15B is a sectional view of the rod of FIG. 15A, taken along section B-B of FIG. 15A.

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

Definitions

As used herein, the singular forms “a” and “the” can include plural referents unless the context clearly dictates otherwise. Thus, for example, reference to “a sponge” can include one or more of such sponges, if the context so dictates.

As used herein, the term “substantially” refers to the complete or nearly complete extent or degree of an action, characteristic, property, state, structure, item, or result. As an arbitrary example, an object that is “substantially” enclosed is an article that is either completely enclosed or nearly completely enclosed. The exact allowable degree of deviation from absolute completeness may in some cases depend upon the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. As another arbitrary example, a composition that is “substantially free of” an ingredient or element may still actually contain such item so long as there is no measurable effect as a result thereof.

As used herein, the term “about” is used to provide flexibility to a numerical range endpoint by providing that a given value may be “a little above” or “a little below” the endpoint.

Relative directional terms can sometimes be used herein to describe and claim various components of the present invention. Such terms include, without limitation, “distal,” “proximal,” “upward,” “downward,” “horizontal,” “vertical,” etc. These terms are generally not intended to be limiting, but are used to most clearly describe and claim the various features of the invention. Where such terms must carry some limitation, they are intended to be limited to usage commonly known and understood by those of ordinary skill in the art in the context of this disclosure. In some instances, dimensional information is included in the figures. This information is intended to be exemplary only, and not limiting. In some cases, the drawings are not to scale and such dimensional information may not be accurately translated throughout the figures.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Invention

As used herein, “minimally invasive” refers to a surgical procedure that is performed through tiny incisions instead of one large opening. During a typical minimally invasive procedure, surgeons make several small incisions in the skin, on the order of a few millimeters, in some cases. There are described devices used in such procedures that can pass through one of the incisions. These devices are called trocars or cannulas. The trocars act as a portal to the body cavity as they traverse into the body cavity.

There are disclosed herein devices used to clean both the trocar and the lens of a scope used in minimally invasive surgery. In an embodiment, the device comprises a rod 14 with cleaning segments or assemblies 10, 11 that may be attached to both ends. The cleaning segments can include, for example, foam or sponges or various components combined into sponge assemblies. The two-headed device can clean a variety of trocar sizes and various scopes. In an embodiment, the minimally invasive device described herein concurrently cleans trocars or cannulas and the lens of the scope.

There is also disclosed a method of cleaning trocars or cannulas of a scope concurrently with the lens of a scope by using the minimally invasive device described herein. In an embodiment, the method comprises cleaning both the trocar and the lens of a scope used in minimally invasive surgery by inserting a rod through the trocar and the foam end conforms to the inner cannula of the trocar. The rod is gently slid through the trocar and debris or fluid is pushed out of the trocar or absorbed by the sponge. Concurrently, the foam end may be used to clean the lens of the scope while the scope is still in the body cavity. Different shapes of foam with varying lengths and diameters are available for both ends of the rod. This variety maximizes the utility of the two-headed rod.

In one embodiment, the method may comprise attaching a first sponge to the rod, inserting one end of the device through the trocar such that at least one sponge is in contact with the inner surface of the trocar. Once the device is engaged within the trocar, the rod is translated and rotated and used to clean the inner cannula. The device is removed after sufficient cleaning is performed. The scope, or other surgical instruments, may then be reinserted through the trocar. If debris or fluid is still within the trocar, the cleaning procedure is repeated. If the lens becomes dirty, the rod with the attached sponge may also be used to enter another trocar and clean the lens of the minimally invasive scope without removing the scope from the body cavity. The sponges are detachable and new sponges may be exchanged multiple times during the procedure, if the sponge becomes dirty or damaged.

A device is described to clean a wide range of trocars regardless of diameter or length. The device includes a rod with two variegated ends. Sponges or a variety of foam shaped structures may be attached and detached to the ends of the rod. These sponges are available in various lengths, widths, and shapes to accommodate a wide range of minimally invasive and robotic trocars and procedures. Once the device is inserted through the trocar, the sponges can be used to clean the scope lens.

Numerous types of sponges may be attached and detached to the rod as needed to ensure that the trocar and lens remain clean during the entire surgical procedure. The scope and trocar cleaning device may consist of a rod with a sponge or foam connected to each end. The figures and text describe the rod and sponge in more detail. The varying degree of sizes and types of sponges allow the device to clean a wide range of trocars while exchanging the foam ends during the procedure if the sponge becomes dirty. Numerous manufacturing companies make a variety of different trocars with varying diameters. Different size sponges may be attached to the rod to accommodate the various trocars. Regardless of size, the same sponge may be used to clean the scope lens.

The rod may be made of a hard plastic, silicone, stainless steel, or various alloys. The rod may be used as a disposable or a reusable device depending on the type of material. In one embodiment, the length of the rod should extend 10 cm, at a minimum. As well, rods may extend over 50 cm for longer trocars.

The rod may have a circular circumference but may also have a hexagonal circumference to enhance tactile feedback and twirling of the device to clean the trocar and lens. Alternatively, various external circumferences or shapes of the rod may be used to augment twirling of the rod as it is inserted and manipulated.

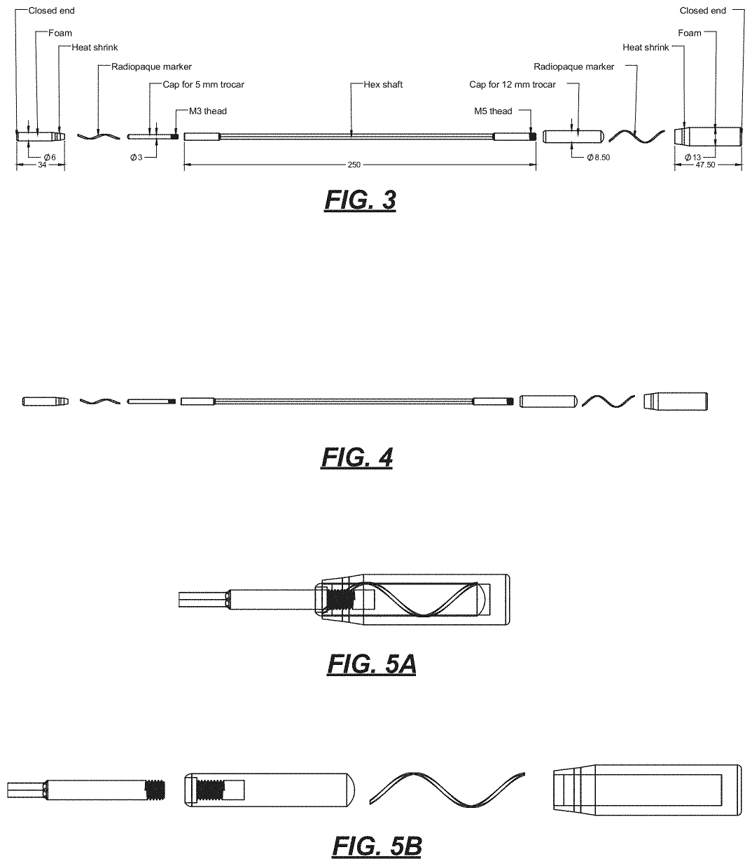

The ends of the rod may include threads, clips, hooks, or variegated edges that facilitate a wide range of sponge attachments. As shown in FIGS. 3, 4 and 5, the ends of the rod may be threaded so sponges may be screwed onto the end of the rod. FIG. 3 particularly demonstrates two different ends of the rod. The ends of the rod may be receptive or threaded. Clips or hooks may be used at the end of the rod and sponges may be hooked or clipped onto the end of the rod. Also, various snaps and matching edges may be used to attach the sponge to the end of the rod. This type of configuration allows new sponges to be used during a single operative procedure.

With further reference to FIG. 3, an X-ray detectable strip may be attached, embedded and/or wrapped around the rod if the rod is made of a material that is not X-ray detectable. In one embodiment, the X-ray detectable strip may consist of a piece of wire or thread that extends the entire length of the rod or along specific segments of the rod. With further reference to this embodiment in FIG. 3, the radiopaque strip that may be positioned between sponge and the trocar to allow the surgeon to visualize the positioning and orientation of lens cleaner when positioned within the body cavity. The outer wall of the trocar may include a groove configured to receive a strip. The groove may have a width and depth substantially equal to or greater than the width and depth of the strip to allow the strip to be recessed into the outer wall of trocar. This position of the strip allows the pad to maintain an unobstructed absorbent surface and prevents any scratching from contact between the strip and the lens of the scope. As depicted in FIG. 3, the strip and groove may each have a helical configuration extending circumferentially around the rod. The helical configuration of the strip allows visualization of the lens cleaner at any angle. However, it is also contemplated that the strip may have other configurations such as linear or serpentine (not shown).

In other embodiments, the strip may be embedded within the sponge. In other embodiments, the strip may be formed from radiopaque markings or dyes applied to the sponge. Additional strips may be provided to allow the surgeon to enhance visualization of lens cleaner through X-ray, fluoroscopy, MRI, and/or CT scan imaging.

In various embodiments, the sponge may be made of foam, cotton, or an absorptive material that can gently clean the lens of a scope. The material should be sturdy enough to clean the inside of the trocar. The sponge may be manufactured into various shapes such as a cylinder, sphere, hexagon, orthotope, rectangular cuboid or ellipse to enhance trocar cleaning.

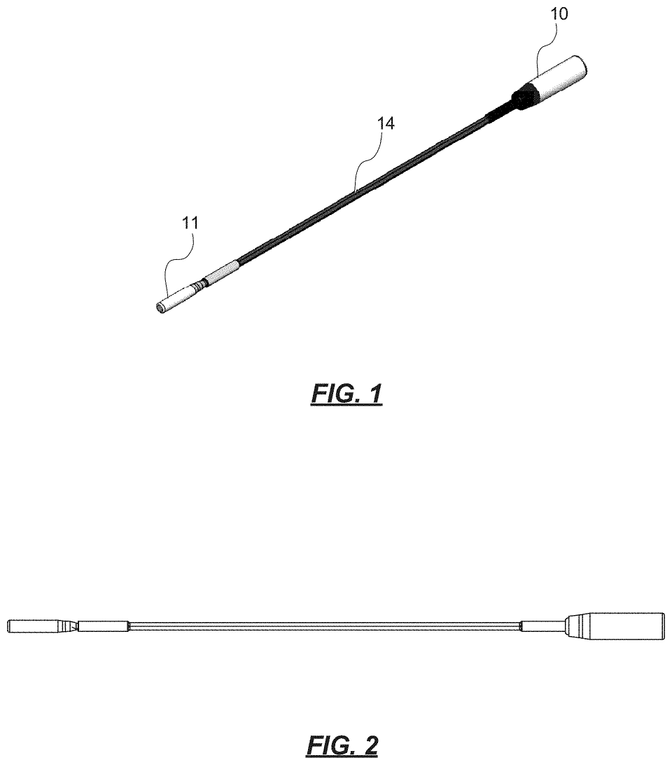

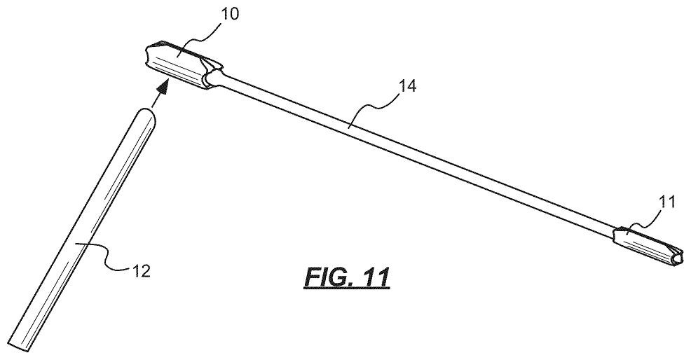

The length and diameter of the sponge includes a range of sizes to accommodate various trocar diameters and lengths. In one embodiment, the sponge is at least 1 cm in length and may extend to 4 cm in length. The diameter of the sponge may range from 2 mm to 50 mm, such as from 5 mm to 40 mm, 10 mm to 30 mm, or 15 to 25 mm. Since the rod entails two ends, two different or two similar sizes of sponge may be attached to each respective end of the rod. FIGS. 1 and 2 show rods with different size sponges and FIGS. 9 and 10 show rods with similar size sponges.

The sponge material can compress as it is inserted through a cannula or trocar. The sponge material would then expand slightly as it exits the trocar and extends into the body cavity. The trocar tip and undersurface of the trocar tip are cleansed as the sponge expands when it exits the trocar and enters the body cavity.

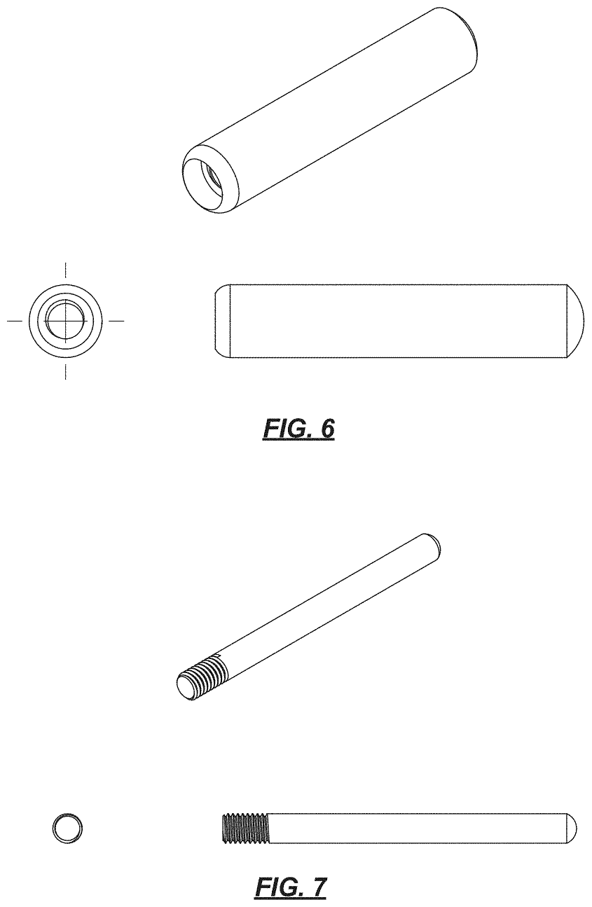

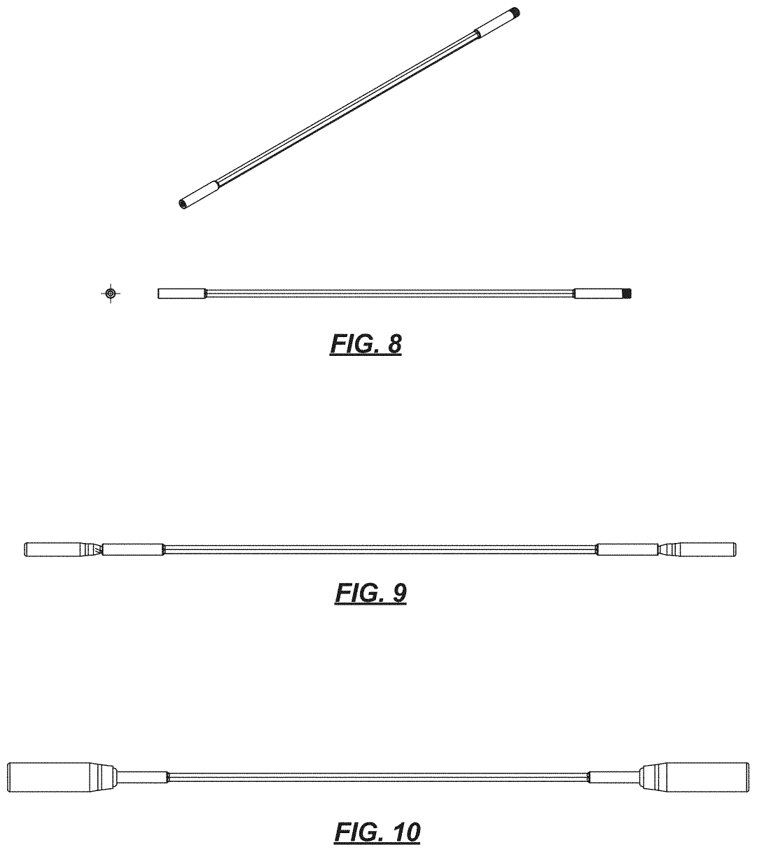

The sponges may have corresponding adaptive couplers, threads, or cored inlets to attach seamlessly to the ends of the rod. The rod may extend into the center of the sponge or entirely through the sponge to ensure adequate support and structural integrity. As shown in FIGS. 5, 6, and 7, the sponge can include a cap that inserts directly into the sponge and then onto the rod as well. As shown in FIGS. 4, 5, 6, and 7, the cap may have a receptive end for a threaded rod or the cap may be threaded to attach to the receptive end of the rod.

An X-ray detectable strip is embedded or wrapped around the sponge. This strip may help locate the sponge if it becomes detached in the body. The strip may be a piece thread, wire, or sheet of X-ray detectable material.

An exemplary method may be disclosed herein. During an exemplary minimally invasive procedure, the lens of the scope inevitably becomes smudged or dirty and obscures the surgeon's view. The scope does not need to be removed from its respective trocar. The two-headed rod is used through a second trocar. The diameter and length of the second trocar is grossly measured and a corresponding sponge is attached to the rod. Two slightly different sponges or similar sponges may be used depending on the trocar or type of debris or fluid on the lens. The unique ability of this device allows the scope lens to be cleaned without removing the scope from the body cavity.

During an exemplary minimally invasive procedure, the device is inserted through a second trocar while the scope remains in the body. The sponge on one end of the rod is inserted through the trocar and directed towards the lens of the scope. Simultaneously, the scope can visualize the device as it enters the body cavity through the tip of the trocar. The scope is held in place but is directed towards the sponge. The sponge is then used to wipe the lens gently to remove fluid, tissue, or condensation from the lens. This action may be repeated multiple times to obtain a clear field of vision.

Alternatively, the rod and corresponding sponge may be held steady and the scope may be advanced slowly towards the sponge. The scope may then be wiped gently against a stationary sponge to clear any debris from the field of vision. The entire device may be removed from the second trocar and flipped 180 degrees along its long axis and the sponge at the opposite end of the rod may be used in a similar fashion to clean the lens of the scope. Additionally, the sponge may be two sided, presenting two different cleaning mechanisms to the scope lens, depending on the orientation of the baton. For example, a first sponge side may have a more porous foam material to aid in removing more difficult debris, while the other side may have a softer, finer pore material to minimize the likelihood of damage to the scope lens. Depending on the cleaning mechanism desired, the surgeon may rotate the baton to introduce one side of the sponge, or the other, to the scope lens. As well, the entire device may be removed from the trocar and the sponge may be removed and replaced with a clean sponge or a sponge with a different shape or contour. The capacity to remove and add new sponges enables the device to continual clean a dirty or smudged lens.

If debris or fluid accumulates within the trocar, the device may be used to clean the inner tunnel of the trocar. The sponge at the tip of the rod is inserted through the center of the trocar and slowly introduced through the entire length of the trocar. The rod may be twirled or twisted as the sponge is inserted to ensure that the entire tract of the trocar is cleaned. The sponge is then removed from the trocar and the scope may be introduced through the clean inner tunnel of the trocar. If the trocar still contains tissue or fluid, the sponge may be reinserted. If the sponge is dirty, then the opposite end of the device may be inserted. Alternatively, the sponge may be detached and a new clean sponge may be attached to the end of the rod.

As depicted herein, the unique capacity of this device embodies both a trocar cleaning device and a device to simultaneously clean a scope lens during a minimally invasive or robotic procedure. The method of cleaning may be performed during any surgery involving a scope. For example, the method may be applicable to procedures performed laparoscopically, thoracoscopically, endoscopically, and robotically.

Other types of cleaning devices that might be in combination with the disclosed device are described in U.S. Pat. No. 10,114,216 (U.S. Published Appl. No. 2016/0022367), which is herein incorporated by reference. The device described in this comprises a sleeve including an inner surface configured to engage a medical device; and a pad secured around the sleeve and configured to wipe the lens of the scope.

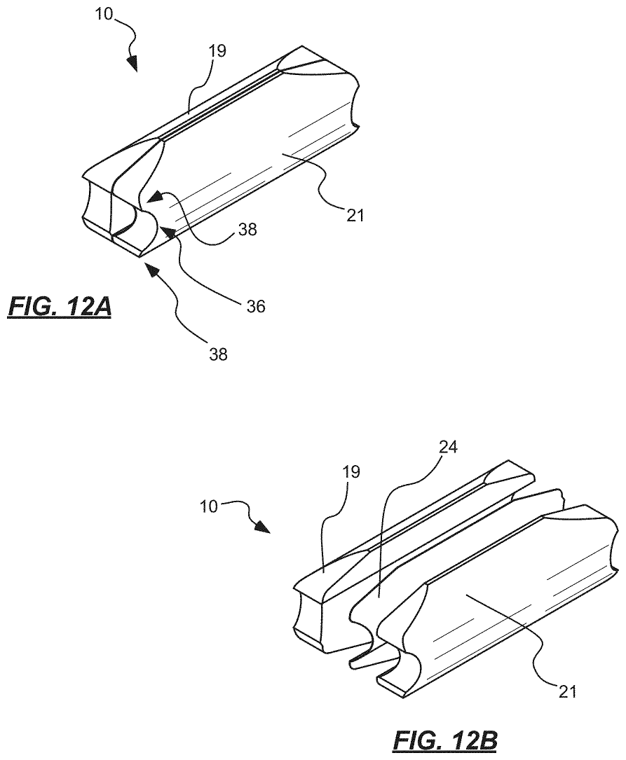

Turning to FIGS. 11 through 15B, further embodiments of the technology are illustrated that include various features that enhance the efficacy of the surgical accessory. In the embodiment of FIGS. 11 through 12B, the surgical accessory or cleaning device includes a rod 14 with a pair of sponge assemblies 10, 11 disposed about ends of the rod. The rod functions in much the same manner as those described above. As depicted, an end of scope 12 can be applied to either of the two sponge assemblies to thereby clean, treat or otherwise affect the scope. The sponge assemblies, when pulled, twisted or pushed through a trocar, also serve to clean the internal portion of the trocar.

In the example shown, a minimally invasive device for cleaning surgical instruments is providing, including a rod 14 having first and second ends, with a first sponge assembly 10 fitted about the first end of the rod. A second sponge assembly 11 can be fitted about the second end of the rod. The sponge assemblies can be secured to or about the rod in a variety of manners. In one example, at least one of the first and second sponge assemblies can be removably secured to one of the respective first and second ends of the rod. As used herein, when a component is “removably” secured or attached or coupled to another component, it is understood that said attachment is accomplished in a such a manner that separation of the two components can be accomplished with damaging either component or without rendering either component unfit for its customary use.

The sponge assemblies can be removably attached to respective ends of the rod to enable a user to easily and apply and remove sponge assemblies. This can advantageously allow users to select a particular size sponge assembly for a specific trocar, and to easily replace used or soiled sponges with clean sponge assemblies. The sponge assemblies can be fitted directly about the rod, as shown in FIGS. 11, 13A and 14A though 14C, or can be fitted about an end cap that can be attached to the rod (see, e.g., end rod 40 in FIG. 15B).

The present technology provides various manners by which sponge assemblies can be removably attached to rods while the sponge assemblies remain suitably secured to the rod to prevent inadvertent movement. In some embodiments, the sponge assemblies can be rotationally retained about the first or second ends. In this manner, the sponge assemblies are restrained from rotating relative to the rod, even in the event the sponge assemblies are not bonded to or otherwise securely fixed to the rod. In this manner, the sponge assemblies can be used to more aggressively clean the internal portions of a trocar, or the lens of a scope, without moving or bunching while doing so. This aspect of the technology can also be beneficial when it is desired to maintain a particular orientation of the sponges relative to the rod during a cleaning or treating process.

This can be advantageous in combination with various embodiments in which the sponge assemblies present two or more distinct cleaning interfaces. This aspect of the technology is illustrated in most detail in FIGS. 12A and 12B. In these figures, sponge assembly 10 is illustrated in further detail having a first cleaning interface, shown generally at 19, and a second cleaning interface, shown generally at 21. Each of these cleaning interfaces can be configured to be contacted by internal portions of a trocar, or a lens or other portion of a scope.

In some embodiments, the first cleaning interface 19 can differ from the second cleaning interface 21 so as to provide at least two differing cleaning interfaces for cleaning the scope 12. The cleaning interfaces can be varied in a number of manners. In one aspect, the cleaning segments are formed from materials that exhibit differing material properties. For example, the first cleaning segment can be formed from a material that is more porous than the second cleaning segment, or more abrasive, softer or harder, tougher, more or less absorbent, hydrophilic vs. hydrophobic, more or less radiopaque, to name a few. A surgeon may find, for example, depending upon the unwanted material that has collected on the scope lens, that the first cleaning segment is better suited to remove fluids or particulates from the scope lens, while the second cleaning segment is better suited to remove vapor deposits from the lens.

The differences in material properties can be accomplished in a variety of manners. For example, the material from which each component is formed can be varied. A manner in which or by which the segments are treated can also be varied. For example, one material can be treated with a finish that affects it toughness, porosity, hardness, etc. The materials may, for example, share the same base structure but differ in some manner. For example, each may be formed from the same polymeric material, but one segment may be provided with a higher degree of porosity. A surface of one or both of the segments may be treated with an additional material that alters the cleaning interface presented by that particular material. For example, a film may be applied to the surface of one of the segments that results in that segment being hydrophobic, while the other segment remains hydrophilic. Any of a variety of known materials and material formation, material finishing techniques, etc., can be utilized to form or alter or condition the differing cleaning interfaces presented.

The manner in which first 19 and second 21 cleaning interface segments are coupled to one another can vary. In the example shown in FIG. 12B, the segments can initially be provided as two distinct components. Outer or side edges of the segments can be coupled one to another. In this manner, a receiving channel is formed as an envelope between the two cleaning segments. This envelope can expand from a very thin slit (as shown in FIG. 12A) to a generally cylindrical opening to receive the rod or other component therein.

The sponge assembly 10 can also include a buffer layer 24 disposed on an inside surface of at least one of the cleaning interface segments. The buffer layer can be positioned so as to contact the rod when the sponge assembly is fitted about the rod. The buffer layer can serve a number of purposes. In one embodiment, the buffer layer presents a coefficient of friction lower than a material of the sponge assembly. This can aid in sliding the sponge assembly over any risers or protrusions included on the rod to retain the sponge assembly in position. The buffer layer can also be formed from a tougher material than the sponge material, to aid in preventing tearing of or damage to the sponge material.

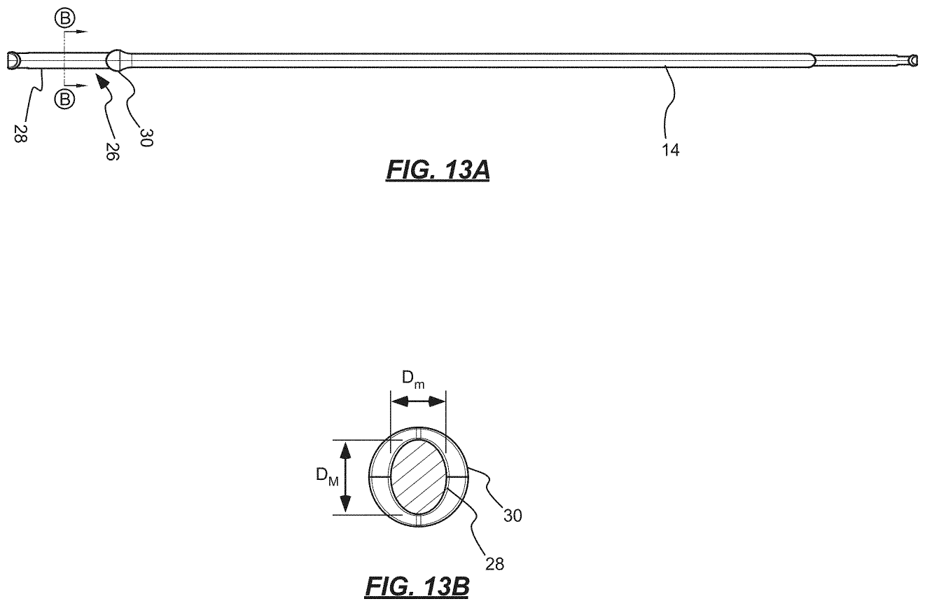

In the embodiment illustrated in FIGS. 13A and 13B, at least one end of the rod 14 can include an indentation 26 sized to receive a sponge assembly therein. The indentation can serve as a detent for the sponge assembly, limiting movement of the sponge assembly in one or more directions. In the example shown, the indentation can include an outer surface 28 recessed relative to an outer surface 30 or protrusion of an adjacent portion of the rod. One or both ends of the indentation can include relatively raised portions or protrusions to retain the sponge or sponge assembly within the indentation. In this manner, the sponge assembly can be restrained from lateral movement relative to the rod.

In one aspect of the technology, the indentation 26 can include a major diameter “D_{M}” and a minor diameter “Dₘ.” The major diameter and minor diameters can cooperate to inhibit rotation of the sponge assembly relative to the rod. The sponge assembly can generally be formed from a pliable material and can include an inner diameter equal to or slightly smaller than the minor diameter of the indentation. The sponge assembly can stretch or extend to fit about the major diameter. When thus positioned about the rod in the indentation, the sponge assembly is restrained from rotating relative to the rod. In this manner, the sponge assembly will not move beneath the forces generated during cleaning of a trocar or a lens of a scope.

The protrusion 30 can include a major diameter larger than an inner diameter of the sponge assembly. In other words, an inner diameter of the sponge assembly can, when in a relaxed condition, be too small to pass over the protrusion 30. When the sponge is formed from a pliable material, however, the sponge can be expanded and extended over the protrusion. After the sponge passes over the protrusion, it will contract into the detent and radially beneath the protrusion: the protrusion then serves to retain the sponge in position laterally.

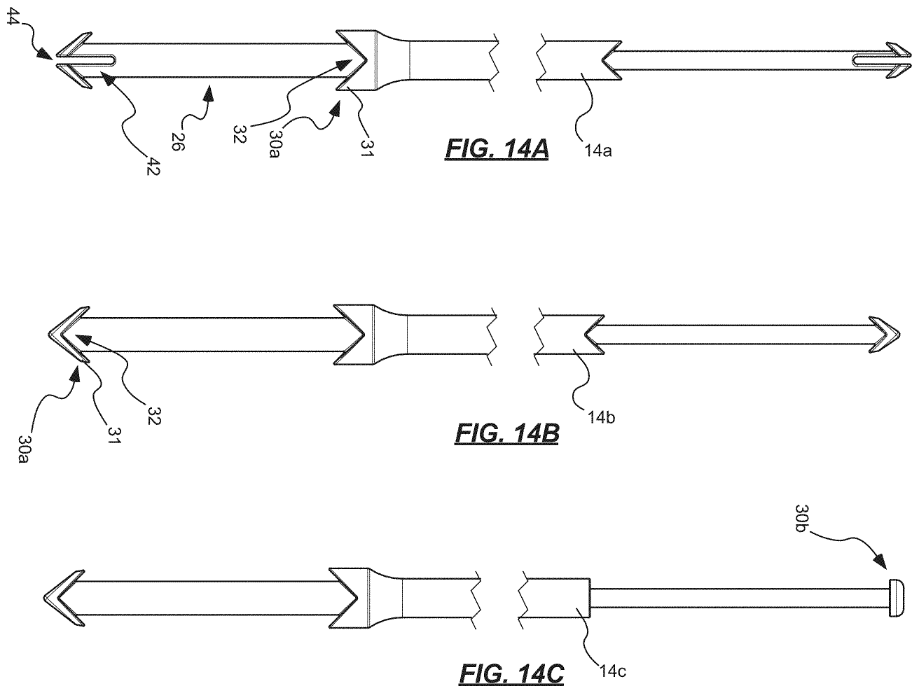

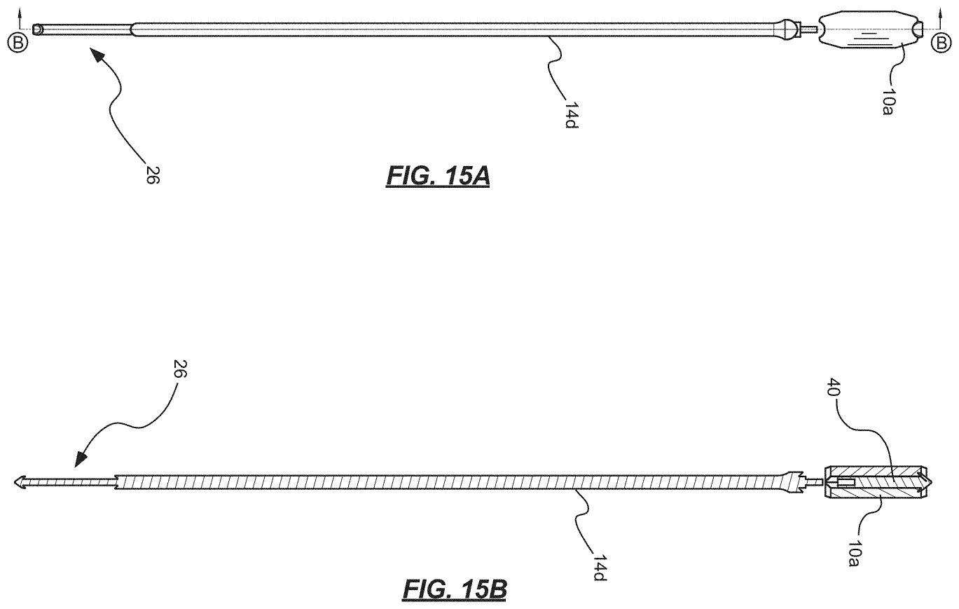

As shown in more detail in FIGS. 14A through 14C, in some embodiments the protrusion 30a, 30b can include a contact profile that engages an adjacent sponge assembly. The contact profile can include one or more walls that extend radially outward from the rod. In the example of FIG. 14C, the wall of protrusion 30b extends radially from the rod in a substantially linear manner. This interface can effectively limit lateral movement of the sponge while providing a smooth transition surface for sliding the sponge assembly over the protrusion. In the examples shown in FIGS. 14A and 14B, the walls can have defined therein at least one notch 31 or peak 32 that are either or both configured to engage an adjacent sponge assembly to thereby inhibit both lateral movement of the sponge assembly and rotation of the sponge assembly relative to the rod.

As shown in FIGS. 12A and 12B, in one embodiment, the sponge assembly 10 can also include a contact profile having a notch 36 and/or peak 38. The notch or peak of the sponge can correspond to the notch 32 or peak 31 of the contact profile of the protrusion 30a. In this manner, the sponge or sponge assembly, after being slid over protrusion 30a, can be snugly fit within the indentation 26 and secured in position as the notch 36 engages peak 31.

In the example shown in FIG. 14A, the ends of the rod can include one or more deformation zones 42 that can compress or plastically deform to allow a sponge assembly to be more easily slid over the protrusion 30a. The deformation zones can include, for example, a slot 44 formed in the rod that defines a pair fingers that can relatively easily be deflected to allow the sponge to be slid over the protrusion. Once released, the fingers will return to a relaxed position and retain the sponge assembly in position relative to the rod.

Much of the structure discussed in relation to FIGS. 13A through 14C includes sponges or sponge assemblies disposed directly about ends of the rod. In other words, internal portions of the sponge can be in direct contact with external portions of the rod. In some embodiments, however, as shown for example in FIGS. 15A and 15B, a sponge assembly 10a can be disposed about a secondary or end rod 40 that can be removably attached to rod 14d. In the example shown, the secondary rod is threadably attachable to the rod 14d. The secondary or end rod, however, can alternately be attached in a variety of suitable manners, such as by a snap connection, a pinned connection, etc.

In addition to the structural components discussed above, the present technology also provides various methods that employ the various components. Suitable methods are provided of manufacturing, using, assembling and positioning devices to clean surgical devices.

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for the features of interest, but not to exclude such from the scope of the disclosure entirely unless otherwise specifically indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

It is to be understood that the above-referenced arrangements are illustrative of the application for the principles of the present invention. Numerous modifications and alternative arrangements can be devised without departing from the spirit and scope of the present invention while the present invention has been shown in the drawings and described above in connection with the exemplary embodiments(s) of the invention. It will be apparent to those of ordinary skill in the art that numerous modifications can be made without departing from the principles and concepts of the invention as set forth in the examples.

We claim:

1. A minimally invasive device for cleaning surgical instruments, comprising:
a rod having first and second ends;
a first sponge assembly fitted about the first end of the rod; and
a second sponge assembly fitted about the second end of the rod;
at least one of the first and second sponge assemblies being removably secured to one of the respective first and second ends of the rod;
each of the first and second sponge assemblies being rotationally retained about the first or second ends of the rod such that the sponge assemblies are restrained from rotating relative to the rod.

2. The device of claim 1, wherein at least one end of the rod includes an indentation sized to receive a sponge assembly therein, the indentation having an outer surface recessed relative to an outer surface of an adjacent portion of the rod, the sponge assembly thereby being restrained from lateral movement relative to the rod.

3. The device of claim 2, wherein the indentation includes a major diameter and a differing minor diameter, the major diameter and minor diameters cooperating to inhibit rotation of the sponge assembly relative to the rod.

4. The device of claim 1, wherein at least one end of the rod includes a protrusion having a major diameter larger than an inner diameter of the sponge assembly, the sponge assembly being restrained by the protrusion from lateral movement relative to the rod.

5. The device of claim 4, wherein the protrusion includes a contact profile, the contact profile including at least one notch or peak that engages the adjacent sponge assembly to thereby inhibit rotation of the sponge assembly relative to the rod.

6. The device of claim 5, wherein the sponge assembly includes a contact profile having a notch or peak corresponding to the notch or peak of the contact profile of the protrusion.

7. The device of claim 1, wherein at least one of the sponge assemblies presents differing first and second cleaning interfaces, each configured to be contacted by the surgical instrument.

8. The device of claim 7, wherein the first and second cleaning interfaces are formed from differing materials.

9. The device of claim 8, wherein a material property of the first cleaning interface differs from a material property of the second cleaning interface in at least one of: a porosity, a toughness, an abrasiveness, a radiopacity and a hardness.

10. The device of claim 1, further comprising a buffer layer disposed on an inside surface of at least one of the sponge assemblies, the buffer layer positioned so as to contact the rod when the sponge assembly is fitted about the rod.

11. A minimally invasive device for cleaning surgical instruments, comprising:
a rod having first and second ends;
a first sponge assembly fitted about the first end of the rod; and
a second sponge assembly fitted about the second end of the rod;
at least one of the first and second sponge assemblies being removably secured to one of the respective first and second ends of the rod; and
at least one end of the rod including an indentation sized to receive one of the sponge assemblies therein, the indentation having an outer surface recessed relative to an outer surface of an adjacent portion of the rod, the sponge assembly thereby being restrained from lateral movement relative to the rod;
each of the first and second sponge assemblies being rotationally engaged about the first or second ends of the rod such that the sponge assemblies are restrained from rotating relative to the rod.

12. The device of claim 11, wherein the indentation includes a major diameter and a differing minor diameter, the major diameter and minor diameters cooperating to inhibit rotation of the sponge assembly relative to the rod.

13. The device of claim 11, wherein at least one end of the rod includes a protrusion having a major diameter larger than an inner diameter of the sponge assembly, the sponge assembly thereby being restrained from lateral movement relative to the rod.

14. The device of claim 13, wherein the protrusion includes a contact profile, the contact profile including at least one notch or peak that engages the adjacent sponge assembly to thereby inhibit rotation of the sponge assembly relative to the rod.

15. The device of claim 5, wherein the sponge assembly includes a contact profile having a notch or peak corresponding to the notch or peak of the contact profile of the protrusion.

16. A minimally invasive device for cleaning surgical instruments, comprising:
a rod having first and second ends;
a first sponge assembly fitted about the first end of the rod; and
a second sponge assembly fitted about the second end of the rod;
at least one of the first and second sponge assemblies being removably secured to one of the respective first and second ends of the rod;
each of the first and second sponge assemblies being rotationally retained about the first or second ends of the rod such that the sponge assemblies are restrained from rotating relative to the rod;
at least one of the sponge assemblies presenting differing first and second cleaning interfaces, each configured to be contacted by the surgical instrument.

17. The device of claim 16, wherein the first and second cleaning interfaces are formed from differing materials.

18. The device of claim 17, wherein a material property of the first cleaning interface differs from a material property of the second cleaning interface in at least one of: a porosity, a toughness, an abrasiveness, a radiopacity and a hardness.

19. The device of claim 16, further comprising a buffer layer disposed on an inside surface of at least one of the sponge assemblies, the buffer layer positioned so as to contact the rod when the sponge assembly is fitted about the rod.

20. The device of claim 19, wherein the buffer layer presents a coefficient of friction lower than a material of the sponge assembly.
